(19) United States
(12) Patent Application Publication
(10) Pub. No.: US 2004/0076604 A1
(43) Pub. Date: Apr. 22, 2004

(54) Tolergenic antigen presenting cells and in treating immune-inflammatory conditions

(76) Inventors: Joan Stein-Streilein, Beverly, MA (US); J Wayne Streilein, Beverly, MA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/468,944

(22) PCT Filed: Feb. 22, 2002
(86) PCT No.: PCT/US02/05441

(51) Int. Cl. A61K 38/19; A61K 45/00

(52) U.S. Cl. 424/85.1; 424/93.7

(57) ABSTRACT

The invention is directed to the preparation and use of tolerogenic antigen presenting cells as a method of down-regulating an immune response in a patient who has an established immune-inflammatory condition or who is at risk of suffering from an immune-inflammatory condition. The tolerogenic antigen presenting cells re-educate patient's own defense cells not only to suppress secretion of cytokines that generates an inflammatory response, but also to stimulate the secretion of immunosuppressive agents to ameliorate debilitating immune effects.

### CROSS REFERENCE TO RELATED APPLICATIONS

This application claims the benefit under 35 U.S.C. §119(e) of U.S. Provisional Patent Application No. 60/270,616 filed on Feb. 22, 2001, entitled EX VIVO THERAPY FOR AUTOIMMUNE AND OTHER DISEASES AND FOR PROLONGATION OF GRAFT SURVIVAL, the whole of which is hereby incorporated by reference herein.

### STATEMENT REGARDING FEDERALLY SPONSORED RESEARCH OR DEVELOPMENT

Part of the work leading to this invention was carried out with United States Government support provided under a grant from the National Institute of Health, Grant Nos. EY11983 and EY13066, and from the National Eye Institute National Research Service Award, Grant No. EY07021-01. Therefore, the U.S. Government has certain rights in this invention.

### BACKGROUND OF THE INVENTION

The process of converting an antigenic signal into an effector immune response in a mammal begins when specific T and B cells recognize antigen on the surfaces of antigen presenting cells (APCs) in the parafollicular regions of the cortex of secondary lymphoid organs. APCs are usually macrophages or dendritic cells, such as Langerhan's cells in the epidermis. Providing that the APCs present antigen plus costimulation, lymphocytes that recognize the antigen are activated. In usual circumstances, the first cells to respond are CD4+ cells. These cells, which recognize antigenic peptides presented in the context of class II MHC molecules, proliferate and secrete cytokines such as IL-2, IL-3, GM-CSF, IFN-γ, and IL-4. In turn, these cytokines act as “helper” factors which “help” the responses of the other cells in the immediate environment. In addition, IL-2, IFN-γ and IL-4 promote the activation and differentiation of CD8+ T cells, which eventually become cytotoxic. CD8+ T cells recognize antigen-derived peptides in the context of class I MHC molecules. Upon antigen-dependent activation, CD8+ T cells not only carry out lytic function, but they also produce cytokines, especially IFN-γ, which have “helper-type” as well as proinflammatory effects on surrounding cells. Finally, the cytokines secreted by CD4+ T cells provide “help” to the activation of B lymphocytes. If the cytokines produced by CD4+ T cells are predominately IFN-γ and IL-2, then the responding B cells produce IgG antibodies that fix complement. Alternatively, if the helper CD4+ T cells produce primarily IL-4, IL-5, IL-6 and IL-10, the responding B cells produce IgG antibodies that do not fix complement, and/or the B cells produce IgE and IgA antibodies.

Thus, central processing allows the antigenic signal received by the lymph node or spleen to be converted into immune effectors, including CD4+ and CD8+ T cells, and B cells secreting a range of different immunoglobulins. All of these effectors share the property of displaying surface receptors that are highly specific for the eliciting antigen. Moreover, as antigen-specific T and B cells proliferate and differentiate in the lymph node or spleen, their progeny, as well as secreted antibody molecules, are delivered into the bloodstream where they are disseminated throughout the circulation. Some of the lymphocytes that emerge from the central processing mechanism will function as effector cells, while others will function as memory cells, having converted into long-lived cells with the capacity to recirculate repeatedly throughout the blood and various tissues.

Hematogenous dissemination of immune effectors generated during central processing makes it possible for these effectors to gain access to all potentially infected tissues and organs served by blood vessels. A typical site of infection or immunization is usually inflamed, and the blood vasculature at the site is usually leaky. Because of this state, and because immune effector T cells display receptors that bind to ligands on activated vascula endothelial cells, immune effectors tend to localize at sites of inflammation, injury, etc. If the relevant antigen is present at the site, then antigen recognition takes place, the effector cells that invade the site are terminally activated, and the final act of immune elimination is triggered. For effector T cells, the ability to recognize antigen at the peripheral site still requires that the antigen be presented by an APC. Professional APC within the inflamed site may serve this function, but often, under the influence of IFN-γ, parenchymal cells (such as epithelial cells, fibroblasts, and even vascular endothelial cells) upregulate class I and class II MHC expression and present antigenic epitopes to T cells. No such requirements exist for specific antibodies that penetrate into the inflamed, antigen-containing site.

While interaction with antigen is the initial step in the process that leads to immune elimination, under most circumstances the actual destruction of the antigen/pathogen is accomplished by the effectors of innate immunity. For example, effector CD4⁺ T cells have little capacity directly to eliminate antigen-bearing target cells. Instead, antigen presentation triggers these cells to secrete lymphokines, such as IFN-γ and TNF-α, which recruit to the site monocyte/macrophages, neutrophils and NK cells. Activation of these cells leads to the generation of cytotoxic products, to the phagocytosis of the offending pathogen, and to activation of the capacity to kill the target cell. In the case of antibody, interactions with antigen and then complement components leads to elimination of the target antigen via direct lysis of cells and by recruitment of neutrophilic granulocytes that destroy the offending pathogen/antigen. Thus, in the expression of immunity, antigen-specific immune effectors usually bring about neutralization and elimination of the invading pathogen by enlisting the aid of the effectors of innate immunity, i.e. nonspecific defense mechanisms.

While elimination of invading pathogens is certainly a desireable capability of an individual's immune system, sometimes, the immune system attacks the individual's own body components generating a debilitating disease or a change in function. For example, multiple sclerosis is a chronic autoimmune disease with symptoms of diminishing muscle control. Asthma or allergic responses to certain foods, grasses, etc., are also undesireable overactive responses of the immune system. Understanding how the response-triggering antigens are initially recognized by APCs may provide insight into ways of controlling this response.

During induction of immunity, i.e. the afferent limb, the key activities are the capturing, processing and presentation of antigen. Since these are largely the functional properties of the APCs, regulation of immune induction is exerted primarily via these cells. Various mechanisms can operate to prevent APCs from presenting antigen to T cells. First, antigen can be sequestered or prevented from gaining access to APCS. Physical barriers can accomplish this, or the APCs themselves (or auxiliary phagocytic and degrading cells) can be rendered incapable of capturing antigen from the microenvironment. Second, APCs can be inhibited from degrading antigen into immunogenic peptides, and/or inhibited from loading these peptides onto MHC molecules in the endoplasmic reticulum, and/or inhibited from expressing peptide-loaded MHC molecules on the cell surface. Third, antigen-bearing APCs may be inhibited from migrating from the somatic tissue of origin to draining lymphoid organs where naïve T cells are first encountered. Fourth, the capacity of APCs to upregulate costimulatory molecules may be suppressed. In general, the inhibitions of APCs function result in a kind of immunologic ignorance in which naïve T cells with specificity for the antigen in question are never confronted by the antigen in immunogenic form.

Other types of APC regulation during immune induction actually lead to “tolerance.” APCs are functionally plastic, and the cytokine milieu in which they reside often dictates the type of functional properties they display. Following high-dose UVB radiation of skin, APCs come under this damaging influence, and begin to secrete IL-10 rather than IL-12. As a consequence, the cells acquire accessory properties that favor the activation of Th2 rather than Th1 cells, i.e., immune deviation. Similarly, APCs within immunosuppressive microenvironments of immune privileged sites (such as the eye) display an unusual array of costimulatory properties, and as a consequence they fail to activate effector CD4⁺ T cells. Instead, they activate regulatory T cells—another form of immune deviation. Finding a way of purposefully altering an undesirable immune response by inducing tolerance in an individual's APCs, thus altering one's own defense cells to control the undesirable immune response, would be valuable.

### BRIEF SUMMARY OF THE INVENTION

This invention is directed to a method of altering the reactivity of a patient's own defense cells by treating antigen presenting cells (APCs) from the patient so as to make them tolerogenic to a specific antigen and then using these tolerogenic APCs to suppress the patient's immune response to the antigen. This tolerance response occurs at sites other than those normally considered to be immune privileged. The method of the invention alters the immune response associated with defense cells to secrete immunosuppressive agents. A process of “re-education” takes place in which the functions of the patient's own defense cells are altered indefinitely. The patient's defense cells are sensitized not only to suppress secretion of cytokines that generate an inflammatory response, but also, to secrete immunosuppressive agents to ameliorate destructive, e.g., autoimmune effects.

The concept of the invention is that a patient to be treated donates, e.g., peripheral blood from which a population of APCs is isolated. These cells are tested for their response to specific antigens that could be responsible for the pathologic condition of the patient. Once an antigen is identified as inducing a positive response, the patient donates, e.g., peripheral blood or bone marrow as a source of a larger quantity of cells, from which APCs, e.g., dendritic cells, are isolated. The APCs are cultured, e.g., in vitro, first with, a cytokine, e.g., TGF-β, and then with the chosed antigen. The treated cells are tested in vitro for their ability to down regulate the immune response at issue. Treated cells showing a positive response as suppressor antigen presenting cells, i.e., they are now tolerogenic, are then re-inoculated into the patient and serve to “re-educate” the patient's resident APCs.

Therefore, in one aspect, the invention is directed to a method of down-regulating an immune response in a patient, wherein the patient has an established immune-inflammatory condition. The method comprises the steps of testing the patient for a response to an antigen. Testing for a positive response to an antigen can be performed using conventional techniques known in the art. Upon a positive response to the antigen, a population of APCs is collected from the patient. The APCs are incubated with an inhibitory cytokine. Then these treated APCs are incubated with the antigen thereby generating tolerogenic APCs. The tolerogenic APCs are treated to a patient for down-regulating an immune response.

In another aspect of the invention, the patient is diagnosed with an immune-inflammatory condition or is at risk of suffering from an immune-inflammatory condition where the antigen relevant to the condition is known. Accordingly, this method down-regulates the patient's immune response who has an established immune-inflammatory condition or who is at risk of suffering from an immune-inflammatory condition. The patient is provided to isolate or collect a population of APCs. The APCs are treated with an inhibitory cytokine by incubation and then incubated with the known antigen, thereby generating tolerogenic APCs. The patient is subsequently treated with these tolerogenic APCs of the invention to down-regulate a debilitating immune response.

In another aspect of the invention, the invention is directed to an immunosuppressive agent for use during an immune-inflammatory condition, wherein the agent comprises a population of antigen presenting cells treated with an inhibitory cytokine and a relevant antigen, whereby the agent alters the reactivity of antigen presenting cells to become re-educated, thereby down-regulating an immune response in a patient with the immune-inflammatory condition.

In a further aspect of the invention, the immunosuppressive agent is used to treat a patient with an immune-inflammatory condition or is at risk of suffering from an immune-inflammatory condition by administering to the patient an effective amount of the immunosuppressive agent of the invention.

The immune-inflammatory condition of the invention includes, but are not limited to, autoimmune conditions and allergic conditions. In one aspect, the autoimmune conditions include, but are not limited to, multiple sclerosis, pulmonary fibrosis, lupus, diabetes. The immune-inflammatory condition of the invention also includes, but are not limited to asthma and possible rejection following grafting, transplantation and xenotransplanation. Exemplary allergic conditions include, but are not limited to allergies against Timothy grass pollen, dander and dust.

The inhibitory cytokine of the invention include, but are not limited to, transforming growth factor-β (TGF-β), which includes TGF-β1 or TGF-β2, interleukin-10 (IL-10), vasoactive intestinal peptide (VIP) and α-melanocyte stimulating hormone (α-MSH).

The APCs of the invention may include, but are not limited to, mature dendritic cells, immature dendritic cells, macrophages and B-cells. The APCs may be collected or isolated from a patient's peripheral blood. In another apsect, APCs may be collected or isolated from a patient's bone marrow.

In another aspect of the method of the invention, the method down-regulates the patient's immune response for at least one year. The method of the invention down-regulates the patient's immune response for at least two years.

In yet another aspect of the invention, a kit for making tolerogenic APCs is provided to treat an immune-inflammatory condition. The kit comprises an inhibitory cytokine, an antigen relevant to the immune-inflammatory condition, and instructions for use in making tolerogenic APCs for the treatment of the immune-inflammatory condition.

The inhibitory cytokines of the kit may include, but are not limited to, TGF-β (TGF-β1 or TGF-β2), IL-10, VIP and α-MSH. The immune-inflammatory condition may be an autoimmune condition that may be, but not limited to, multiple sclerosis, pulmonary fibrosis, lupus, diabetes, asthma, graft survival, transplantation and xenotransplanation. The immune-inflammatory condition may also be an allergic condition that reacts against Timothy grass pollen, dander or dust.

The kit of the invention may treat, for example, multiple sclerosis by including all the components for making tolerogenic APCs. The components may include, but are not limited to, TGF-β in tolerogenic buffer, myelin basic protein (MBP), and instructions for use in making tolerogenic APCs for the treatment of multiple sclerosis.

### BRIEF DESCRIPTION OF THE DRAWINGS

Other features and advantages of the invention will be apparent from the following description of the preferred embodiments thereof-and from the claims, taken in conjunction with the accompanying drawings, in which:

FIGS. 1A-1B is a comparison of MBP-induced EAE disease progression in C57BL/6 wild-type and Jα281^{-/-} mice. FIG. 1A shows mean clinical EAE scores of all mice in WT vs. Jα281^{-/-} groups over time is given. FIG. 1B shows percent incidence among all mice over time in WT vs. Jα281^{-/-} groups over time is given. (N=5 per group.) Data shown are representative of two experiments in which similar results were obtained;

FIGS. 2A-2B show Effects of NK1.1⁺ cell reconstitution on EAE development in NKT cell-deficient mice. FIG. 2A shows NK1.1⁺ cells were enriched by magnetic cell separation. Dot blots show the magnitude of enrichment of cells that express TCRβ chain and NK1.1, both at intermediate density. The percentage of NKT and NK cells before and after enrichment is shown beneath the dot blots. FIG. 2B shows a line chart indicates the mean clinical EAE scores of all mice in groups of B6 WT vs. Jα281^{-/-} mice vs. NKT-reconstituted Jα281^{-/-} mice. (N=5 per group.) Data shown are representative of two experiments in which similar results were obtained;

FIG. 3 shows the effects of Ag-pulsed, tolerogenic APC treatment on the development of EAE in conventional C57BL/6 mice. Mice were given 1.0×10⁶ immunogenic (MBP-pulsed) or tolerogenic (MBP+TGFβ-pulsed) APCs i.v., seven days after receiving immunization with MBP. Clinical EAE scores for individual mice in each group is shown. (N=10 for untreated and immunogenic group, N=9 for tolerogenic group). Data shown are compiled from two experiments in which similar results were obtained. The horizontal line indicates the mean for the group;

FIGS. 4A-4C show the effects of Tr cell transfer on the induction of EAE in naïve C57BL/6 mice. FIG. 4A shows dot blots represent the degree of enrichment of total splenic T cells and CD4 and CD8-depleted fractions prior to being transferred to naïve mice that were subsequently immunized with MBP. FIG. 4B shows a line chart represents mean EAE clinical scores over time among all mice in groups of B6 mice given no cells, enriched T cells from immunogenic or tolerogenic APC-treated mice, and CD4 and CD8-depleted T cell fractions from tolerogenic APC-treated mice. FIG. 4C shows a bar graph of the ear swelling (DTH) response to OVA of naïve B6 mice immunized with OVA in CFA (s.c.) and given OVA-pulsed PECs intradermally to the ear pinnae (pos DTH) vs. CD8⁺ Tr cell recipients (MBP-tolerant mice). Naïve B6 mice that received only ear challenge with OVA-pulsed PECs served as a negative DTH control;

FIG. 5 shows a bar graph of a hydroxyproline analysis of lungs of Hapten Immune Pulmonary Interstitial Fibrosis (HIPIF) mice treated with tolerogenic antigen presenting cells (APCs);

FIG. 6 shows a bar graph of contact hypersensitivity ear swelling analysis of mice received enriched T cells from tolerogenic dendritic cell-treated HIPIF mice;

FIG. 7 shows a bar graph of whole body Barometric Plethysmography measurement of airway hyperresponsiveness (AHR) in OVA-asthma mice that are treated with Anterior Chamber-associated Immune Deviation-inducing dendritic cells; and

FIG. 8 shows a RT-PCR analysis of IL-4, IL-5, IL-13 and IFN-γ mRNA levels in bronchoalveolar lavage (BAL) cells.

### DETAILED DESCRIPTION OF THE INVENTION

This invention is directed to a method of altering the reactivity of a patient's own defense cells by creating and then using tolerogenic antigen presenting cells (APCs) so as to suppress the patient's immune response to an immune-inflammatory condition at sites other than those normally considered to be immune privileged. The defense cells used in the present invention may comprise immature dendritic cells, mature dendritic cells, macrophages and B-cells. The method of the invention alters the immune response associated with defense cells to not only suppress the secretion of cytokines that generate an inflammatory response, but also to stimulate the secretion of immunosuppressive agents. Tolerogenic antigen presenting cells created according to the invention re-educate the function of the patient's own defense cells so as to elicit a different immune response. This method is not only effective as a prophylactic but also effective in treating established autoimmune or allergic conditions.

Tolerogenic APCs are modified according to the invention with suppressive cytokines. Exemplary cytokines include, but are not limited to, transforming growth factor-β (TGF-β1 or TGF-β2), α-melanocyte stimulating hormone (α-MSH), interleukin-10 (IL-10), and vasoactive intestinal peptide (VIP). Appropriate amounts can readily be determined by conventional protocols. Exemplary dosages of inhibitory cytokines may range anywhere from about 30 μg per 50 μl of supernatant or buffer to about 5 ng per ml of supernatant or buffer for, e.g., 2×10⁵ to 4×10⁵ dendritic cells. Optimal incubation periods may range, for antigen presenting cells and an inhibitory cytokine, anywhere from about 6 hours to about 24 hours. Optimal incubation periods for the treated antigen presenting cells with a relevant antigen may range from about 2 hours to about 24 hours.

The treatment with tolerogenic APCs of the invention allows for a patient to remain tolerant for at least one year. In one aspect, such treatment re-educates a patient's defense cells for at least two years. In another aspect, the treatment re-educates a patient's defense cells indefinitely. There seems to be no potential danger from the procedure if the cells are maintained according to sterile and clinical standards.

In accordance with the invention, the patient to be treated donates, e.g., peripheral blood cells or bone marrow cells, which contain the defense cells to be “re-educated.” The defense cells are tested first, in vitro, for their response to potential/relevant antigen that is relevant to the pathological condition of the patient. A response to an antigen may be determined by conventional protocols, for example, by various markers or antibodies. If a positive response is obtained to one or more antigen, a second population of cells from, e.g., peripheral blood or bone marrow from the patient is obtained so that a large quantity of APCs can be obtained. Appropriate quantities can readily be determined standard protocols. While APCs can be grown and expanded using conventional methods, about 10 to 30 million peripheral blood cells per 10 ml of blood, for example, may be appropriate. Exemplary amounts for APCs may range from about 2×10⁶ to about 5×10⁶ cells. The APCs are cultured from about 6 hours to about 24 hours, in vitro, with an inhibitory cytokine. Subsequently, the APCs treated with the inhibitory cytokine is incubated with an antigen for about 2 hours to about 24 hours; this treatment alters the manner in which these cells present antigen to the defense cells.

This is tested by exposing the original defense cells to the treated APCs and determining whether the former respond in a typical fashion. If the response of the defense cells is altered, then the treated APCs are judged to be appropriately altered as well. By administering these tolerogenic APCs into the blood stream of the patient, a process of re-education takes place in which the patient's own defense cells' functions are permanently altered. In mice, as few as 20 tolerogenic APCs are sufficient to achieve this re-education.

The immune-inflammatory condition of the invention includes, but are not limited to, autoimmune conditions and allergic conditions. In one aspect, the autoimmune conditions include, but are not limited to, multiple sclerosis, pulmonary fibrosis, lupus, diabetes. The immune-inflammatory condition of the invention also includes, but are not limited to asthma and possible rejection following grafting, transplantation and xenotransplanation. Exemplary allergic conditions include, but are not limited to allergies against Timothy grass pollen, dander and dust.

### EXAMPLES

Exemplary applications in accordance with the present invention are described in the following disease conditions: experimental autoimmune encephalomyelitis (EAE) in mice (EAE is a mouse model for multiple sclerosis in humans), pulmonary fibrosis, asthma and allergy to Timothy grass pollen. But, the invention is not intended to be limited to these disease conditions.

The following examples are presented to illustrate the advantages of the present invention and to assist one of ordinary skill in making and using the same. These examples are not intended in any way otherwise to limit the scope of the disclosure.

### Exemplary Materials and Methods Applicable to the Disease Conditions Described Herein

Mice: Female C57BL/6 mice used in these experiments were obtained from the Schepens Eye Research Institute Vivarium or Taconic Farms (Taconic, N.Y.). Jα281^{-/-} on the C57BL/6 background and CD1d^{-/-} (B6×129, F2) mice were obtained from Drs. Mark Exley and Steve Balk, (Beth Israel Deaconess Medical Center, Harvard Medical School, Boston, Mass.). Mice were housed on a 12/12 h-light/dark cycle and provided food and water ad libitum. All animals were treated humanely and in accordance with the guidelines set forth by the Schepens Eye Research Institute Animal Care and Use Committee and the National Institute of Health (NIH) guidelines.

Induction and measurement of disease:

EAE was induced with an adaptation of the method of Betelli et al (Bettelli et al., 1998). Briefly, mice were immunized subcutaneously (s.c.) on each flank with 200 μg (400 μg total) of myelin basic protein (Sigma Chemical Co., St. Louis, Mo.) in complete Freund's adjuvant (CFA) (Sigma Chemical Co.) containing 4 mg/ml Mycobacterium tuberculosis (H37ra) (Difco Laboratories, Detroit, Mich.). Additionally, all mice received intraperitoneal (i.p.) injections of 200 ng of Bordetella pertussis toxin (Ptx) in PBS on days zero and two (400 ng total). Evaluation of EAE was done for thirty three days using the following clinical scores: 0, no overt signs of disease; 1, limp tail or hind limb weakness; 2, limp tail and hind limb weakness; 3, partial hind limb paralysis; 4, complete hind limb paralysis; 5, moribund or death.

Pulmonary fibrosis was tested using a mouse model for pulmonary interstitial fibrosis in humans. Hapten Immune Pulmonary Interstitial Fibrosis (HIPIF) condition was generated in mice by an allergic contact hypersensitivity response against hapten (TNBS, 2,4,6-trinitrobenzene sulfonic acid) modified self-antigens in the lung.

Mouse OVA-asthma model was initiated by a Th2 mediated immune response in the lung.

NKT cell enrichment and reconstitution in EAE Model: NKT cell enrichment was done as previously described (Sonoda et al., 1999). Briefly, erythrocyte free splenocyte suspensions were prepared from unmanipulated C57BL/6 mice and immunostained with FITC-conjugated anti-NK1.1 mAb (clone PK136, prepared by our laboratory). After thorough washing, the immunostained cells were incubated with anti-FITC magnetic microbeads (Miltenyi Biotec, Auburn, Calif.) and the NK1.1⁺ cells were isolated by passage through MiniMacs™ MS⁺ magnetic separation columns (Miltenyi Biotec). Enrichment of NK1.1⁺ cells was confirmed by flow cytometry. For reconstitution of NKT cells in Jα281^{-/-} mice, NK1.1⁺ enriched cells were treated with DNase (to prevent clumping), washed twice in HBSS, and adjusted to a concentration of 1.0×10⁷ cells per ml of HBSS and each mouse was given 100 μl (1.0×10⁶ cells) via the tail vein.

Treatment with tolerogenic APCs: Tolerogenic APCs were prepared by a modification of methods reported by Streilein and colleagues (Wilbanks et al., 1992). Briefly for the cell-based treatment of EAE mice, thioglycollate-elicited peritoneal exudate cells (PECs) were cultured overnight in serum free medium (SFM) containing 500 μg/ml of bovine MBP (Sigma Chemical) and 5 ng/ml of transforming growth factor beta (TGFβ) 2 (R&D Systems Inc., Minneapolis, Minn.). Control (immunogenic) APCs were prepared in the same manner, excluding the TGFβ2. After culture, the APCs were washed three times in HBSS to remove excess TGFβ and MBP. The APCs were placed at 4° C. in PBS for two hours and collected by vigorous pipetting. The cells were treated with DNase, washed twice and re-suspended at a concentration of 5×10⁶/ml in HBSS. For cell-based treatment of EAE, each mouse was given 100 μl of cell suspension (5×10⁵ cells) via the tail vein seven days after immunization with MBP in CFA.

In the Hapten Immune Pulmonary Interstitial Fibrosis model, tolerogenic dendritic cells were used on the development of fibrosis in mice.

In the mouse OVA-asthma model, tolerogenic dendritic cells were used on the development of Th2 pathogenesis.

Preparation of Tr cell subsets for treatment of EAE: Thirty-three days after treatment of EAE mice with immunogenic vs. tolerogenic APCs, their spleens were collected and processed into erythrocyte-free single cell suspensions. To enrich Tr cells, splenocytes were passed through IMMULAN™ goat anti-mouse IgG columns (Biotecx Laboratories, Houston, Tex.). Enrichment efficiency was confirmed by flow cytometric analysis of negatively selected cells. Depletion of either CD8⁺ or CD4⁺ Tr cells was achieved by incubation of IMMULAN-enriched fractions with either anti-CD8 (ATCC clone 2.43) or anti-CD4 ascites (ATCC clone GK1.5) plus LowTox™ baby rabbit complement (Cedar Lane Laboratories, Hornby, Ontario). Depletion of CD8 or CD4 subsets was confirmed by flow cytometric analyses of cells immunostained with FITC-conjugated anti-CD8 mAb (Ly-2, clone 53-6.7) and PE-conjugated anti-CD4 mAb (L3T4, clone RM4-5) (both from BD Pharmingen, San Diego, Calif.). For treatment with enriched cells, naïve C57BL/6 mice were given 5×10⁶ of the respective cell type (i.v.) suspended in 100 μl of HBSS. Immediately after transfer of cells, Tr cell-treated mice were immunized with MBP in CFA plus Ptx and monitored for clinical symptoms for thirty-three days.

Disease progression:

In the EAE model, mice were inoculated (s.c.) with OVA in CFA (150 μg in 150 μl) and seven days later, received an intradermal injection in the ear pinnae (ear challenge) of 2.0×10⁵ (in 10 μl HBSS) PECs that had been cultured overnight with OVA (5 mg/ml in SFM). Mice that received ear challenge without prior s.c. sensitization served as a negative Delayed Type Hypersensitivity (DTH) control. Differences in DTH responses between the groups were determined by measuring ear thickness before challenge, twenty-four, and forty-eight hours after challenge with an engineer's micrometer (Mitutoyo, Paramus, N.J.).

In the HIPIF model, mice were inoculated with hapten (2,4,6-trinitrobenzene sulfonic acid (TNBS)) modified self-antigens in the lung. Hydroxyproline deposition in the lungs were compared to the HIPIF mice without treatment. As well, contact hypersensitivity ear swelling analysis was also performed after enrichment of T cells from the HIPIF mice treated with or without tolerogenic dendritic cells and transferred (i.v.) to mice that were pre-sensitized with TNBS.

In the mouse OVA-asthma model, an immune response in the lung was mediated by Th2 cytokine (IL-4,5,13). Airway hyperresponsiveness (AHR), a hallmark of asthma, was measured using whole body Barometric Plethysmography in OVA-asthma mice that were treated with Anterior Chamber-Associated Immune Deviation(ACAID)-inducing dendritic cells. Barometric Plethysmography detects enhanced pause (Penh, a parameter of airway resistance) in airways.

Statistical analyses: In the EAE model, statistical differences in the average day of onset and mean peak clinical scores between control vs. experimental groups was determined by either student's T test (depending on the number of groups in the experimental design) or analysis of variance (ANOVA), followed by Neuman Keuls post-hoc analyses. Significance was considered at P<0.05. Statistical differences in DTH responses were determined by student's T test.

### Example I

### Tolerogenic APCs to Generate CD8+ Regulatory T Cells to Ameliorate NKT Cell-Induced EAE

TABLE 1
Comparison of EAE disease parameters
in wild type vs. NKT-deficient mice

| | | | | Average Day |
|---|---|---|---|---|
| | Inci- | | Mean Peak | of Disease |
| Strain | dence | Mortality | Disease Score | Onset |
| C57BL/6 WT | 10/10 | 0/10 | 2.7 ± 0.3 | 9.8 ± 1.9 |
| B6 Jα281^{-/-} | 6/10 | 0/10 | 0.9 ± 0.3* | 18.5 ± 1.8* |
| B6x129 WT | 5/6 | 1/6 | 3.2 ± 0.5** | 3.8 ± 0.5 |
| B6X129 CD1d^{-/-} | 2/4 | 0/4 | 1.5 ± 0.4** | 9.0 ± 2.8** |

*p ≦ 0.05 vs. C57BL/6 WT
**p ≦ 0.05 vs. B6x129 WT

NKT cells contribute to the development of MBP-induced EAE

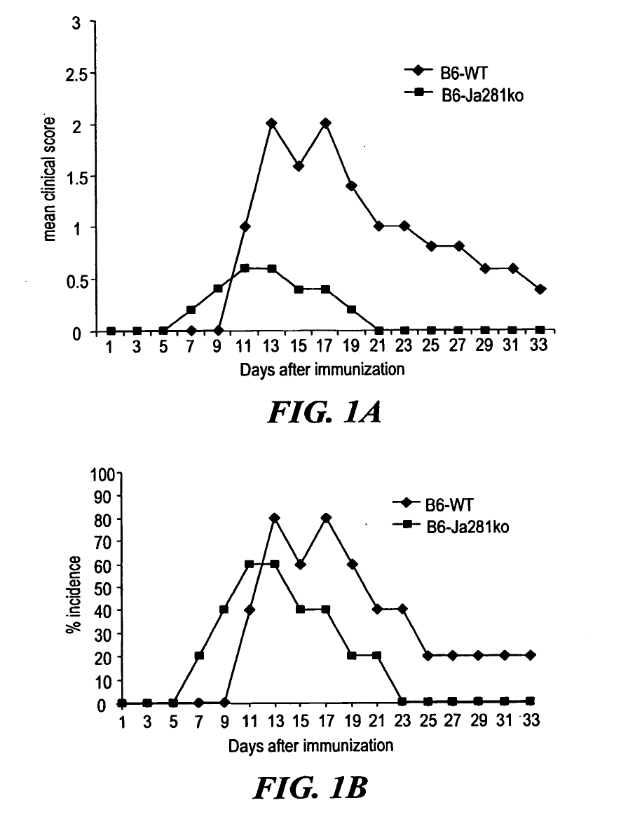

Wild-type C57BL/6 mice (B6-WT) and NKT-deficient Jα281^{-/-} mice (B6 background) were immunized with bovine myelin basic protein (MBP) in complete Freund's adjuvant (CFA) and monitored for the development of EAE symptoms over the course of thirty-three days. Nine days after immunization, B6-WT mice developed modest EAE, with a mean severity score of 2.7±0.3 and an average day of onset of 9.8±1.9. (FIGS. 1A and B, Table 1). In contrast, NKT-deficient Jα281^{-/-} mice had a mean severity score of 0.9±0.3 with an average day of onset of 18.5±1.8. There was a trend towards decreased incidence of disease in the Jα281^{-/-} mice, but the difference between groups was not statistically significant. There was no mortality associated with the MBP-immunization protocol.

TABLE 2
Comparison of EAE disease parameters after reconstitution
of NKT-deficient mice with NK1.1⁺ cells^{a}

| | | | | Average Day |
|---|---|---|---|---|
| | Inci- | | Mean Peak | of Disease |
| Strain | dence | Mortality | Disease Score | Onset |
| C57BL/6 WT | 5/5 | 0/5 | 2.4 ± 0.2 | 9.8 ± 1.9 |
| B6 Jα281^{-/-} | 3/4 | 0/4 | 1.5 ± 0.5* | 18.3 ± 4.6* |
| B6 Jα281 + NKT | 5/5 | 0/5 | 2.6 ± 0.6** | 7.0 ± 0.6** |
| reconstitution | | | | |

^{a}data represent two experiments in which similar results were obtained
*p ≦ 0.05 vs. C57BL/6 WT
**p ≦ 0.05 vs. Jα281 ko

Confirmation of the role for NKT cells in the development of EAE

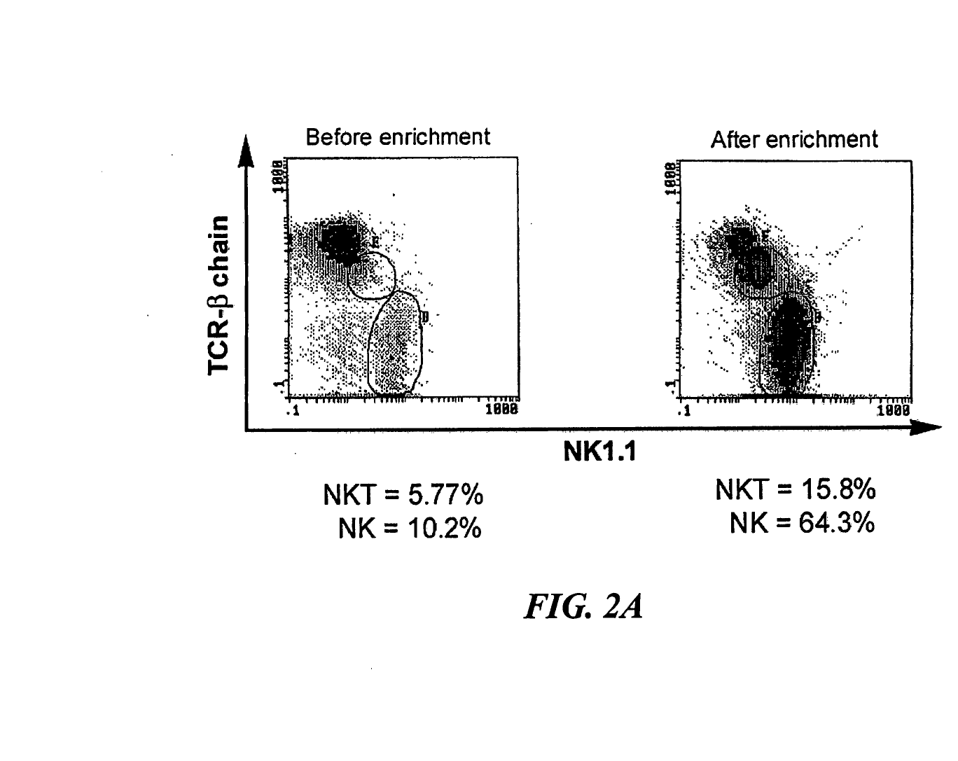

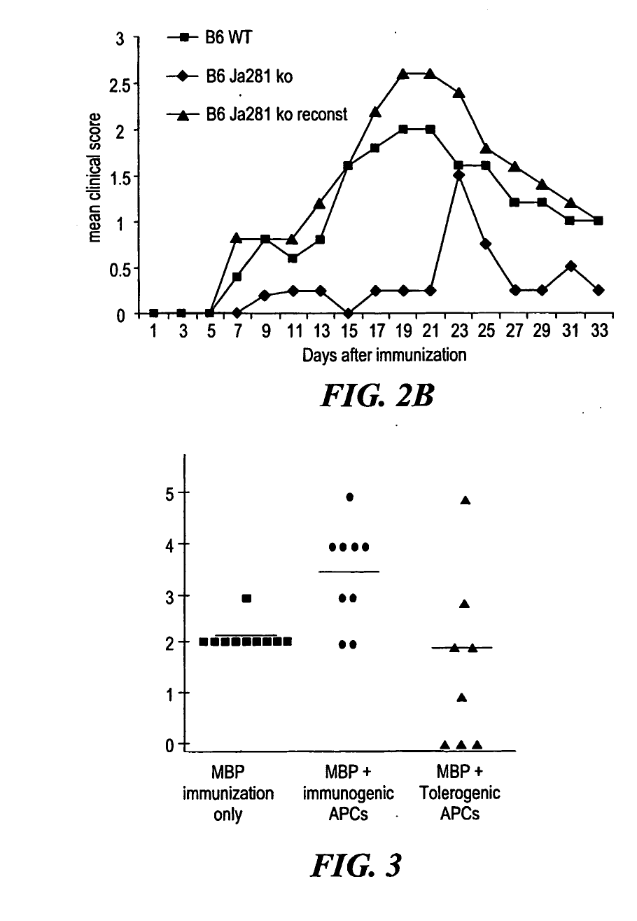

To confirm that NKT cells play a central role in the development of EAE, Jα281^{-/-} mice was reconstituted with NKT cells from B6-WT mice and then tested for the induction of EAE. NK1.1⁺ cells used for reconstitution were enriched from the spleens of naïve B6-WT mice by immunostaining and magnetic cell separation (FIG. 2A). Each Jα281^{-/-} mouse was re-constituted with 10⁶ enriched NK1.1⁺ cells, given i.v. One week later, all mice were immunized with MBP and EAE progression of the groups (B6-WT vs. Jα281^{-/-} vs. NK1.1⁺ reconstituted Jα281^{-/-} mice) was compared.

As before, EAE in Jα281^{-/-} mice was delayed in onset and less severe than in B6-WT mice (FIG. 2B). However, EAE in NKT cell-reconstituted Jα281^{-/-} mice was similar to B6-WT mice (FIG. 2B, Table 2). Therefore, NKT cells contribute to the expression of EAE.

TABLE 3
Effects of immunogenic vs. tolerogenic APCs cell transfer
on ongoing EAE^{c}

| | | | | Average Day |
|---|---|---|---|---|
| | | | Mean Peak | of Disease |
| Strain | Treatment | Incidence | Disease Score^{a} | Onset^{b} |
| C57BL/6 | None | 10/10 | 2.2 ± 0.2 | 9.8 ± 0.9 |
| C57BL/6 | Immunogenic | 9/9 | 3.2 ± 0.5* | 11.6 ± 1.2* |
| | APC | | | |
| C57BL/6 | Tolerogenic | 6/9 | 2.0 ± 0.7** | 14.3 ± 1.7** |
| | APC | | | |

^{a}data are expressed as mean peak disease score ± SEM based on mice that acquired disease
^{b}data are expressed as average day of onset ± SEM
^{c}data are compiled from two experiments in which similar results were obtained
*p ≦ 0.05 vs. untreated
**p ≦ 0.05 vs. Immunogenic APC-treated

The NKT cell's role in EAE development can be influenced by different types of APCs

The fact that NKT cells are necessary for induction of EAE suggests that they promote the generation of an inflammatory T cell response. On the other hand, it was found that when stimulated by Ag-pulsed, TGFβ2-treated APCs, NKT cells promote tolerance. Specifically, when tolerogenic APCs were given to mice in vivo or used in co-culture systems in vitro, they promoted the NKT-dependent generation of CD8⁺ Tr cells that suppressed Ag-specific DTH (Sonoda et al., 1999; Sonoda et al., 2001; Wang et al., 2001). Based on the reasoning from this model, the protocol to induce disease (i.e., Ag, CFA, pertussis) generated inflammatory APCs that in turn influenced the NKT cells to support the immune response. Thus, the present study postulated that adoptive transfer of “tolerogenic” APCs might alter the phenotype of the inflammatory NKT cells towards a tolerogenic or regulatory one.

Immunogenic and tolerogenic APCs were established by culturing thioglycollate elicited peritoneal exudate cells (PECs) in serum free medium containing either MBP (immunogenic) or MBP+TGFβ2 (tolerogenic). APCs of either type were injected (i.v., 10⁶ cells/mouse) into mice immunized with MBP in CFA seven days earlier. A group of mice that received only MBP/CFA immunization (no cell transfer) served as a control. Mice that received MBP immunization plus treatment with tolerogenic APCs exhibited a significant delay in disease onset (day 15 vs. day 9 in controls) (Table 3) and a significantly lower peak EAE score (FIG. 3 and Table 3). While both the untreated control and immunogenic APC-treated groups had 100% incidence of disease (10/10 and 9/9, respectively), it was observed that the tolerogenic APC-treated group had lower incidence (6/9) (Table 3 and FIG. 3). Moreover, the peak EAE scores of mice in the tolerogenic APC-treated group segregated into two groups (FIG. 3), one having either little or no disease (score of 0-1, four mice), the other having moderate to severe disease (score of 2-5, five mice). The fact that the tolerogenic APC-treated group had mice with peak scores of only 0-1 and lower incidence, indicated suppression of EAE due to the tolerogenic APC treatment. Thus, treatment with antigen pulsed, tolerogenic APCs one week after immunization with MBP in adjuvant suppressed the development and severity of EAE, and delayed its onset.

Tolerogenic APCs suppress EAE through the development of CD8⁺ Tr cells that prevent Th1 immunity

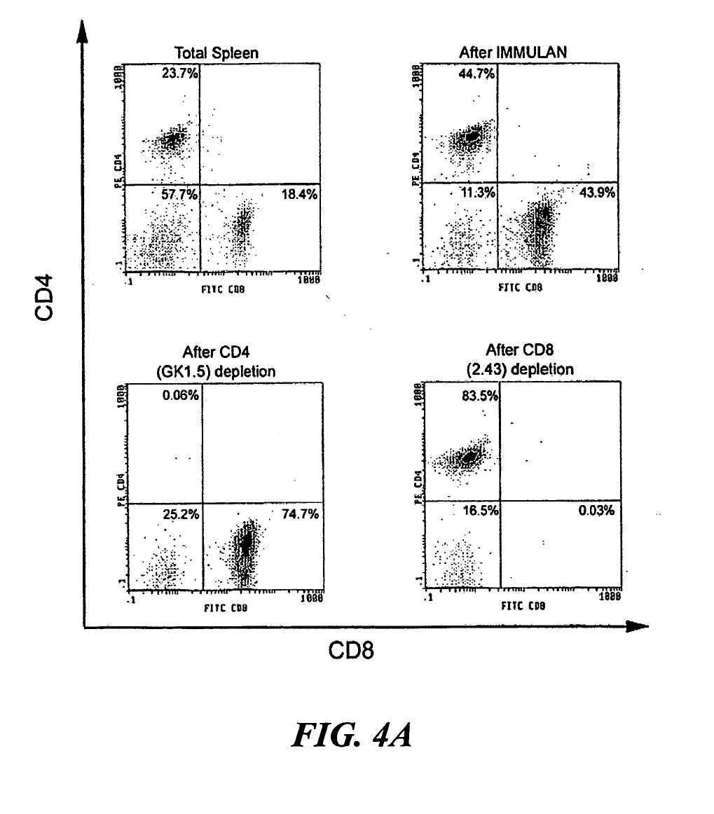

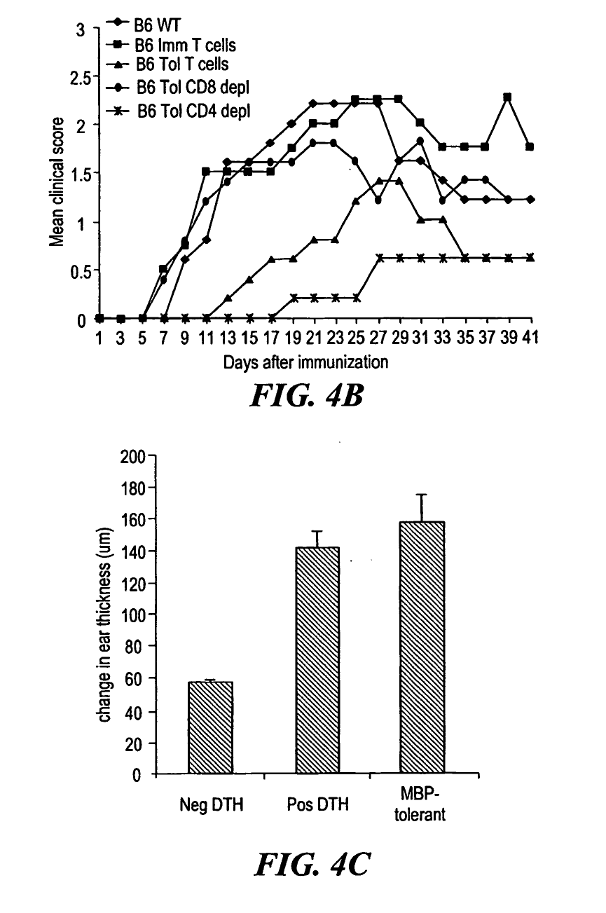

The fact that a subset of tolerogenic APC-treated mice exhibited peak EAE scores of zero or one suggested that EAE was reduced due to the generation of regulatory T cells. Therefore, enriched splenic T cells was prepared from the spleens of tolerogenic APC-treated mice that exhibited a peak EAE score of zero or one (day 33 after MBP immunization), and transferred them to naïve, syngeneic recipients (5×10⁶ cells per mouse). The recipient mice were then immunized with MBP in CFA and monitored for EAE. To determine whether the Tr cell was CD4⁺ vs. CD8⁺, parallel groups of mice were given 5×10⁶ enriched T cells that were depleted of either CD4⁺ or CD8⁺ cells by specific antibody and complement (FIG. 4A). B6 mice that were immunized with MBP in CFA, but received either no cells or T cell transfer from immunogenic APC-treated EAE mice, were used as controls.

MBP-immunized B6 mice that received either no cells or enriched splenic T cells from immunogenic APC-treated mice developed typical EAE (FIG. 4B). In contrast, mice that received enriched T cells from experimental mice (i.e., recipients of tolerogenic APCs) exhibited less severe and delayed EAE compared to the control groups (FIG. 4B). Most notably, adoptive transfer of enriched CD8⁺ (but not CD4⁺) T cells was even more effective in lessening the severity of EAE and delaying its onset. Thus, even in the presence of an ongoing immune response, treatment with tolerogenic APCs promotes the generation of a CD8⁺ Tr cell that suppresses autoimmunity. Moreover, the CD8⁺ Tr cell itself was sufficient to prevent the induction of autoimmunity in naïve mice.

Antigen specificity of the regulation of EAE in mice that received CD8⁺ Tr cells was tested by co-immunization (s.c.) of CD8⁺ Tr cell recipients (MBP-tolerant) at day 33 with a third-party antigen (OVA in CFA). One week later, all mice were challenged in their ear pinnae with OVA-pulsed PECs (2.0×10⁵ cells per ear pinna) and DTH was assessed twenty-four hours later. Unmanipulated B6 mice that received either OVA in CFA (s.c.) plus ear challenge or ear challenge alone, served as positive and negative DTH controls, respectively. While MBP-specific EAE remained suppressed in CD8⁺ Tr cell recipients, the DTH response towards OVA was intact, evidenced by a positive ear swelling response (FIG. 4C). Thus, the prevention of EAE induction by the adoptive transfer CD8⁺ Tr cells was antigen-specific.

Based on the foregoing experiments, NKT cells are required for the development of MBP-induced EAE in conventional C57BL/6 mice. Mice genetically deficient in either NKT cells or CD1d failed to develop EAE with the same magnitude, incidence, and clinical course, as the wild-type controls. The role for CD1d-restricted NKT cells in development of disease was firmly established by experiments in which NKT-deficient mice (Jα281^{-/-}) reconstituted with wild-type NKT cells, developed ascending paralysis that was comparable with control mice. From the present results, in this B6 mouse model of MBP-induced EAE, CD1d-restricted NKT cells supported the Th1 immune response against myelin antigens, rather than protect from it. However, treatment of EAE mice with tolerogenic APCs promoted the differentiation of CD8⁺ Tr cells and the induction of NKT-dependent tolerance. As a result of the treatment, ongoing EAE was ameliorated and disease induction in naïve mice was prevented.

Under inflammatory conditions influenced by NKT cell-autoimmune response in the CNS, immune inflammation can be overcome by the introduction of tolerogenic APCs that convert the phenotype of the NKT cells to one that promotes the generation of antigen specific CD8+ Tr cells. The tolerogenic APCs of the invention stimulated the secretion of immunosuppressive agents. Thus, the present findings show that cell-based therapeutics that modulate NKT cell function and promote tolerance can be used to prevent the progression or recurrence of multiple sclerosis in humans.

### Example II

### Tolerogenic APCs for Suppression of Fibrosis in HIPIF

Pulmonary fibrosis is a debilitating disease in which a chronic inflammatory response leads to permanent fibrosis of lung tissue. Hapten Immune Pulmonary Interstitial Fibrosis (HIPIF), a mouse model for human interstitial pulmonary fibrosis, is elicited by a contact hypersensitivity immune response against hapten (2,4,6-trinitrobenzene sulfonic acid (TNBS)) modified self antigen in the lung. Mice that are pretreated with altered antigen presenting cells (APCs) failed to develop pulmonary fibrosis when they were subsequently exposed to an experimental regimen designed to cause the disease. Moreover, defense cells harvested from these mice similarly prevented pulmonary fibrosis when injected in naïve mice.

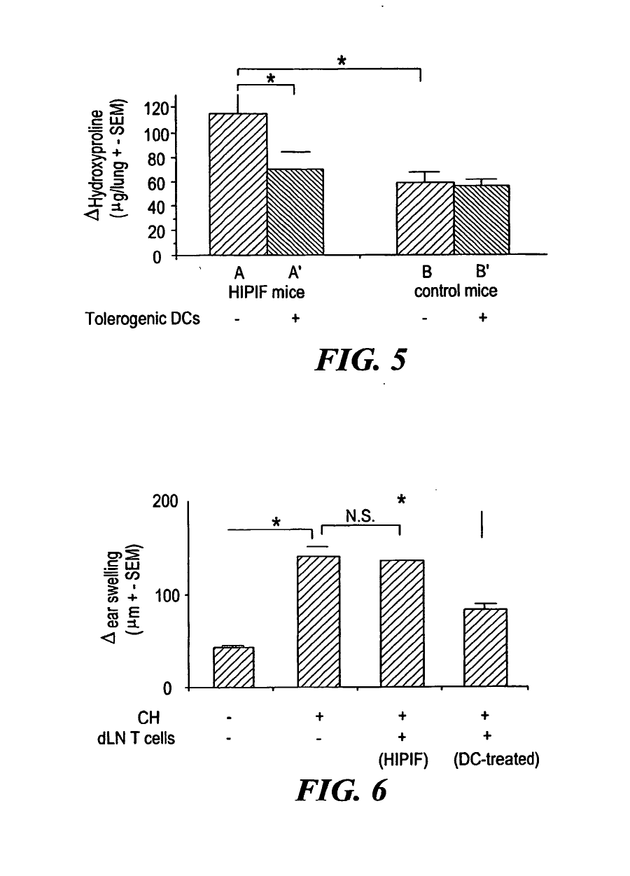

Intravenously (i.v.) inoculated tolerogenic peritoneal exodate cells (PECs) (TGFβ-treated antigen-pulsed) have been known to generate regulatory T (Tr) cells and mediate peripheral tolerance in mice. Obtaining antigen presenting cells from the peritoneal cavity is only appropriate in a murine model. The present study was designed to determine whether induction of peripheral tolerance would down regulate the secondary immune response in the lung and block the development of HIPIF. TGFβ-treated TNBS-pulsed PECs were used to induce tolerance in an adoptive transfer model of HIPIF (ADT-HIPIF). Recipient mice that received TNBS-sensitized cells and challenged intratracheally (i.t.) with hapten developed fibrosis (increased hydroxyproline deposition). However, recipient mice treated with tolerogenic APC (i.v.) one day before i.t. challenge had reduced hydroxyproline accumulation in the lung. Hydroxyproline analysis of lungs of HIPIF mice treated with tolerogenic dendritic cells (DCs) is shown in FIG. 5. The histogram shows the change (Δ) in hydroxyproline content in the lungs of experimental mice. The treatment for each group of mice is described under the abscissa. TGFβ2-treated, TNBS-pulsed (tolerogenic) DCs (5×105/mouse) were transferred (R.O.) to recipient mice one day after i.t. challenge. Lungs were harvested 14 days after i.t. challenge and analyzed for their hydroxyproline content. Asterik (*) indicated significant (p<0.05) difference between two groups that were compared. HIPIF mice received (i.v.) tolerogenic DCs after antigen inoculation into the lung had reduced hydroxyproline deposition (fibrosis) in their lungs compared to the HIPIF mice without the treatment.

As shown in FIG. 6, tolerogenic DCs that were transferred into the HIPIF mice generated efferent T regulatory cells that suppressed an ear swelling contact hypersensitivity (CH) response in pre-sensitized mice. The histogram shows the change in ear swelling of experimental mice. Lung draining lymph node T cells were enriched from the HIPIF mice treated with or without tolerogenic DCs and transferred (i.v.) to mice that were presensitized with TNBS. One day after the transfer, the mice were challenged with TNBS on their ears. The ear thickness was measured 24 hours after ear challenge. Asterisk (*) indicates significant difference between two groups that are compared.

In addition, when the recipient mice received Tr cells harvested from mice that were treated with tolerogenic APC seven days earlier, they did not develop fibrosis. In conclusion, TGFβ-treated antigen-pulsed PECs induce Tr cells that are able to limit the development of autoimmune pulmonary fibrosis in sensitized mice after challenge. Tolerogenic APCs may be a potential therapeutic approach for immune mediated pulmonary diseases and for some forms of idiopathic pulmonary fibrosis in humans.

### Example III

### Tolerogenic APCs for Suppression of Th2-Mediated Pathogenesis in Mouse OVA-Asthma

This study was performed to test the immunobiological therapy for airway hyperreactivity in mice, which is representative mouse model of asthma in humans. Mice acquire airway hyperreactivity, increased mucus and leukocytic infiltrates when first immunized with ovalbumin, then challenged in the airways with the same antigen. This type of airway hyperreactivity is abolished in mice if prior to immunization the mice receive either an anterior chamber injection of OVA, or an intravenous injected of altered antigen presenting cells (APCs) bearing OVA. It was found that tolerogenic APCs blocked Th2 pathogenesis in the mouse OVA-asthma model.

OVA was injected into the anterior chamber (AC) of BALB/c mice to induce ACAID. Seven and twelve days later, the mice were sensitized i.p. with OVA precipitated with aluminum hydroxide. The mice were then challenged intratracheally with OVA nineteen days after AC injection. Three days after the OVA challenge, bronchoalveolar lavage (BAL) fluid was removed, cytospum onto microscope slides, and stained with HEMA 3 stain set for differential cell counts. To analyze cytokine production (by ELISA and RT-PCR), BAL fluid and regional LN cells were collected one day after intratracheal OVA installation. OVA-specific IgE levels were assayed in sera collected seventeen days after AC injection.

Adoptive transfer of tolerogenic dentric cells (DCs), but not OVA-pulsed DCs without TGF-β2 treatment, into presensitized mice, reduced mucus production in the asthmatic lungs and blocked cell (eosinophils, lymphocytes, and monocytes) infiltration into the lungs of OVA-asthma mice (data not shown).

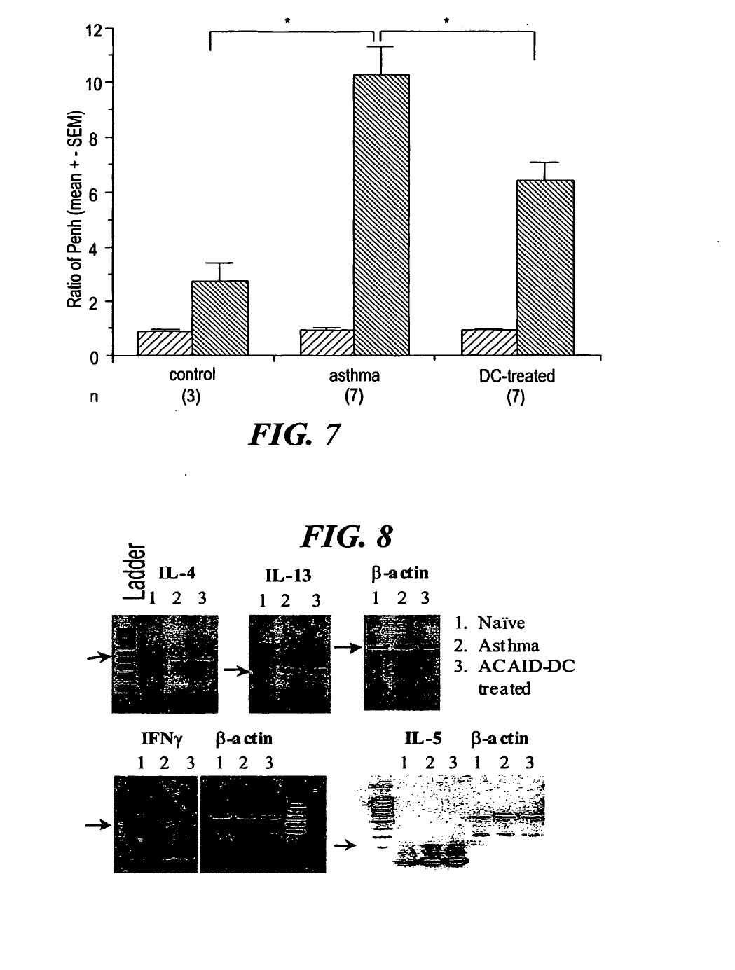

As shown in FIG. 7, tolerogenic DC treatment blocked airway hyperresponsiveness (AHR) (a hallmark of asthma) that was measured using whole body Barometric Plethysmography that detects enhanced pause (Penh, a parameter of airway resistance) in airways. Enhanced Pause (Penh—a parameter of AHR) was measured after mice were aerosolized with either PBS or methacholine (200 mg/ml) and compared among naïve mice, asthma mice, and tolerogenic DC treated asthma mice. The experimental groups and the number of mice in each group are indicated under each bar on the abscissa. The ordinate indicates the ratio of Penh when mice were exposed to either PBS (hatch bar) or methacholine (solid bar) over their baseline (without any stimulation) Penh. Asterisk (*) indicates significant difference between two groups that are compared.

As shown in FIG. 8, tolerogenic DC treatment reduced Th2 cytokine (IL-4,5,13) mRNA levels, but not IFN-y mRNA levels in bronchoalveolar lavage cells from the asthma mice. Panels show the RT-PCR analyses of IL-4, IL-5, IL-13 and IFN-γ mRNA levels in BAL cells collected from naïve (lane 1), asthma (lane 2), or tolerogenic DC-treated asthma (lane 3) mice. RNA used in the RT-PCR analyses was purified from BAL cells collected from a mouse three days after i.t. challenge.

### Example IV

### Tolerogenic APCs for Suppression of an Allergic Response in HU-SCID Mice

Blood from patients with an allergy to Timothy grass pollen is inoculated into the SCID mouse. Because the mouse has no defense cells, leukocytes from the patients survive. When these mice are challenged with grass pollen, they have an allergic response in the lung similar to the response in humans. The same patients' blood antigen presenting cells is treated with TGF-β and Timothy grass pollen and then the cells are injected into HU-SCID mice that previously received untreated patient cells. No pulmonary allergic response is observed.

### USE

The method of the invention has been shown to lead to the reversal of symptoms in mouse models of human disease and other pathological conditions that are mediated by CD4⁺ T cells. These mouse models are considered by those of skill in the art to be directly relevant to the comparable human pathologic conditions. Therefore, the method of the invention is considered to be useful for the reversal of symptoms of human disease and other pathological conditions that are mediated by CD4⁺ T cells. Therefore, the method of the invention is directed to using an individual's own antigen presenting cells (APCs) that have been made tolerogenic in accordance to the invention to an antigen related to their pathological or immune-inflammatory condition.

To test whether a patient is responding to a given antigen, one of ordinary skill in the art can use conventional techniques flow cytometry and chromatographic binding studies to identify a positive result known in the art such as by identification with markers or relevant antibodies.

In the method of the invention, an appropriate amount of of APCs to collect from a patient with an established immune-inflammatory condition or a patient who is at risk of suffering from an immune-inflammatory condition is about 2 to 5 million. These numbers are not definitive since one of ordinary skill in the art would be able to determine appropriate amounts to use depending on the patient and the condition. But, approximately 10 ml of blood, for example, is collected to obtain about 10 to 30 million peripheral blood cells from which the APCs may be isolated.

Exemplary inhibitory cytokines may include, but are not limited to, TGF-β (TGF-β1 or TGF-β2), interleukin-10, vasoactive intestinal peptide, and α-melanocyte stimulating hormone. Exemplary dosages of inhibitory cytokines may range anywhere from about 30 μg per 50 μl of supernatant or buffer to about 5 ng per ml of supernatant or buffer for, e.g., 2×10⁵ to 4×10⁵ dendritic cells. Optimal incubation periods may range, for antigen presenting cells and an inhibitory cytokine, anywhere from about 6 hours to about 24 hours. Optimal incubation periods for the treated antigen presenting cells with a relevant antigen may range from about 2 hours to about 24 hours.

The therapeutic agents of the invention may be administered. intravenously or intra-arterially by routine methods in, e.g., pharmaceutically acceptable inert carrier substances. In one aspect, administration is by any mode deliverable directly into the patient's blood system. For example, the compositions of the invention may be administered in a sustained release formulation using a biodegradable biocompatible polymer, or by on-site delivery using micelles, gels or liposomes. The tolerogenic antigen presenting cells can be administered in a pharmaceutically acceptable inert carrier substance in a dosage of at least 20 tolerogenic APCs. In one aspect, an amount of 10,000 to 20,000 tolerogenic APCs may be appropriate for a human. In another aspect, an amount of 20,000 to 40,000 tolerogenic APCs may be appropriate for a human. Optimal dosage and modes of administration can readily be determined by conventional protocols. Tolerogenic APCs may also be administered with other inhibitors or reducers of inflammation.

The method of the invention may be used to treat various immune-inflammatory conditions. The immune-inflammatory condition of the invention includes, but is not limited to, autoimmune conditions and allergic conditions. In one aspect, the autoimmune conditions include, but are not limited to, multiple sclerosis, pulmonary fibrosis, lupus and diabetes. The immune-inflammatory condition of the invention also includes, but are not limited to asthma, and possible rejection following grafting, transplantation and xenotransplanation. Exemplary allergic conditions include, but are not limited to allergies against Timothy grass pollen, dander and dust. However, the treatment in accordance with the invention most preferably occurs when the patient is in, e.g., remission or not in an acute inflammatory state, or when the patient's inflammatory responses are quiescent. Otherwise, the patient's resident immunogenic APCs can reduce the effects of tolerogenic APCs of the invention.

### REFERENCES

Bendelac, A., M. N. Rivera, S. H. Park, and J. H. Roark. 1997. Mouse CD1-specific NK1 T cells: development, specificity, and function. Annu. Rev. Immunol. 15:535-562.

Bettelli, E., M. P. Das, E. D. Howard, H. L. Weiner, R. A. Sobel, and V. K. Kuchroo. 1998. IL-10 is critical in the regulation of autoimmune encephalomyelitis as demonstrated by studies of IL-10- and IL-4-deficient and transgenic mice. J. Immunol. 161:3229-3306.

Bonish, B., D. Jullien, Y. Dutronc, B. B. Huange, R. Modlin, F. M. Spada, S. A. Porcelli, and B. J. Nickoloff. 2000. Overexpression of CD1d by keratinocytes in psoriasis and CD1d-dependent IFN-y production by NK-T cells. J. Immunol. 165:4076-4085.

BuBhoff, U., A. Hein, A. Iglesias, R. Dorries, and A. Regnier-Vigouroux. 2001. CD1 expression is differently regulated by microglia, macrophages and T cells in the central nervous system upon inflammation and demyelination. J. Neuroimmunol. 113:220-230.

Chen, H. and W. E. Paul. 1997. Cultured NK1.1⁺ CD4⁺ T cells produce large amounts of IL-4 and IFNγ upon activation by anti-CD3 or CD1. J. Immunol. 159:2240-2249.

de Camargo Furtado, G., D. Olivares-Villagomez, M. A. Curotto de Lafaille, A. K. Wensky, J.-A. Latkowski, and J. J. Lafaille. 2001. Regulatory T cells in spontaneous autoimmune encephalomyelitis. Immunol. Rev. 122-134.

D'Orazio, T. J., E. Mayhew, and J. Y. Niederkorn. 2001. Ocular immune privilege promoted by the presentation of peptide on tolerogenic B cells in the spleen. II. evidence for presentation by Qa-1. J. Immunol. 166:26-32.

Eberl, G. and H. R. MacDonald. 1998. Rapid death and regeneration of NKT cells in anti-CD3epsilon- or IL-12-treated mice: a major role for bone marrow in NKT cell homeostasis. Immunity 9:345-53.

Falcone M., Yeung B., Tucker L., Rodriguez E. , and Sarvetnick N. 1999. A defect in interleukin 12-induced activation and interferon gamma secretion of peripheral natural killer T cells in nonobese diabetic mice suggests new pathogenic mechanisms for insulin-dependent diabetes mellitus. J. Exp. Med. 190:963-972.

Faunce, D. E., K.-H. Sonoda, and J. Stein-Streilein. 2001. MIP-2 recruits NKT cells to the spleen during tolerance induction. J. Immunol. 166:313-321.

Gombert, J. M., A. Herbelin, E. Tancrede-Bohin, M. Dy, M. Carnaud, and J. F. Bach. 1996. Early quantitative and functional deficiency of NK1⁺-like thymocytes in the NOD mouse. Eur. J. Immunol. 26:2989-2998.

Inobe, J., A. J. Slavin, Y. Komagata, Y. Chen, L. Liu, and H. L. Weiner. 1998. IL-4 is a differentiation factor for transforming growth factor-beta secreting Th3 cells and oral administration of IL-4 enhances oral tolerance in experimental allergic encephalomyelitis. Eur. J. Immunol. 28:2780-2790.

Jahng, A. W., I. Maricic, B. Pedersen, N. Burdin, O. Naidenko, M. Kronenberg, Y. Koezuka, and V. and Kumar. 2001. Activation of natural killer T cells potentiates or prevents experimental autoimmune encephalomyelitis. J. Exp. Med. 194:1789-1799.

Jiang, H., N. S. Braunstein, and B. Yu. 2001. CD8+ T cells control the TH phenotype of MBP-reactive CD4+ T cells in EAE mice. Proc. Natl. Acad. Sci. USA 98:6301-6306.

Joyce, S. 2001. CD1d and natural T cells: how their properties jump-start the immune system. Cell. Molec. Life Sci. 58:442-469.

Kaneko, Y., M. Harada, T. Kawano, M. Yamashita, Y. Shibata, F. Gejyo, T. Nakayama, and M. Taniguchi. 2000. Augmentation of Vα14 NKT cell-mediated cytotoxicity by interleukin 4 in an autocrine mechanism resulting in the development of concanavalin A-induced hepatitis. J. Exp. Med. 191:105-114.

Koh, D.-R., A. Ho, A. Rahemtulla, J. Penninger, and T.-W. Mak. 1994. Experimental allergic encephalomyelitis (EAE) in mice lacking CD4+ T cells. Eur. J. Immunol. 24:2250-2253.

Kosiewicz, M. M. and J. W. Streilein. 1996. Intraocular Injection of Class II-Restricted Peptide Induces an Unexpected Population of CD8 Regulatory Cells. J. Immunol 157:1905-1912.

Kumar, V. and E. Sercarz. 1998. Induction or protection from experimental autoimmune encephalomyelitis depends on the cytokine secretion profile of TCR peptide-specific regulatory CD4 T cells. J. Immunol. 161:6585-6591.

Lehuen, A., O. Lantz, L. Beaudoin, V. Laloux, C. Carnaud, A. Bendelac, J. F. Bach, and R. C. Monteiro. 1998. Overexpression of natural killer T cells protects Valpha14—Jalpha281 transgenic nonobese diabetic mice against diabetes. J. Exp. Med. 188:1831-1839.

Lesage, S. and C. C. Goodnow. 2001. Organ-specific autoimmune disease: a deficiency of tolerogenic stimulation. J. Exp. Med. 194: F31-F36.

Matsuda, J. L., O. V. Naidenko, L. Gapin, T. Nakayama, C.-R. Wang, Y. Koezuka, and M. Kronenberg. 2000. Tracking the response of natural killer T cells to a glycolipid antigen using CD1d tetrameters. J. Exp. Med. 192:741-753.

Matsumoto, Y., S. Abe, M. Tsuchida, H. Hiraharra, T. Abo, T. Shin, N. Tanuma, T. Kojima, and Y. Ishihara. 1996. Characterization of CD4⁻CD8⁻ T cell receptor αβ⁺ T cells appearing in the subarachnoid space of rats with autoimmune encephalomyelistis. Eur. J. Immunol. 26:1328-1334.

Matsumoto, N., R. K. Ribaudo, J. P. Abastado, D. H. Margulies, and W. M. Yokoyama. 1998. The lectin-like NK cell receptor Ly-49A recognizes a carbohydrate-independent epitope on its MHC class I ligand. Immunity 8:245-54.

Mieza, M. A., T. Itoh, J. Q. Cui, Y. Makino, T. Kawano, K. Tsuchida, T. Koike, T. Shirai, H. Yagita, A. Matsuzawa, H. Koseki, and M. Taniguchi. 1996. Selective reduction of V alpha 14⁺ NK T cells associated with disease development in autoimmune-prone mice. J. Immunol. 156:4035-4040.

Miyamoto, K., S. Miyake, and T. Yamamura. 2001. A synthetic glycolipid prevents autoimmune encephalomyelitis by inducing Th2 bias of natural killer T cells. Nature 413:531-534.

Nickoloff, B. J., B. Bonish, B. B. Huang, and S. A. Porcelli. 2000. Characterization of a T cell line bearing natural killer receptors and capable of creating psoriasis in a SCID mouse model systeme. J. Dermatol. Sci. 24:212-225.

Okamoto, S., M. M. Kosiewicz, R. R. Caspi, and J. W. Streilein. 1994. ACAID as a potential therapy for establishmental autoimmune uveitis. In Advances in Ocular Immunology. R. B. Nussenblatt, S. M. Whitcup, R. R. Caspi, and I. Gery, editors. Elsevier Science, Amsterdam. 195-198.

Olivares-Villagomez, D., A. K. Wensky, Y. Wang, and J. J. Lafaille. 2000. Repertoire requirements of CD4⁺ T cells that prevent spontaneous autoimmune encephalomyelitis. J. Immunol. 164:5499-5507.

Roncarolo, M.-G. and M. K. Levings. 2000. The role of different subsets of T regulatory cells in controlling autoimmunity. Curr. Opin. Immunol. 12:676-683.

Sakaguchi, S. 2000. Animal models of autoimmunity and their relevance to human diseases. Curr. Opin. Immunol. 12:684-690.

Samoilova, E. B., J. L. Horton, and Y. Chen. 1998. Acceleration of experimental autoimmune encephalomyelitis in interleukin-10-deficient mice: Roles of intereleukin-10 in disease progression and recovery. Cell. Immunol. 188:118-124.

Shevach, E. 2000. Regulatory T cells in autoimmunity. Ann. Rev. Immunol. 18:423-449.

Shevach, E. M., R. S. McHugh, C. A. Piccirillo, and A. M. Thornton. 2001. Control of T-cell activation by CD4+ CD25+ suppressor T cells. Immunol. Rev. 182:58-67.

Sonoda, K.-H. and J. Stein-Streilein. 2001. A critical role for CD1d expression by “eye-derived” APCs and splenic marginal zone B cells in the promotion of immune privilege and tolerance. J. Immunol. In press.

Sonoda, K.-H., D. E. Faunce, M. Taniguchi, M. Exley, S. Balk, and J. Stein-Streilein. 2001. NKT cell-derived IL-10 is essential for the differentiation of antigen-specific T regulatory cells in systemic tolerance. J. Immunol. 166:42-50.

Sonoda, K.-H., M. Exley, S. Snapper, S. Balk, and J. Stein-Streilein. 1999. CD1 reactive NKT cells are required for development of systemic tolerance through an immune privileged site. J. Exp. Med. 190:1215-1225.

Steinman, L. 2001. Multiple Sclerosis: a two-stage disease. Nature Immunol. 2:762-764.

Streilein, J. W. 1999. Immunoregulatory mechanisms of the eye. Prog. Retinal Eye Res. 18:357-370.

Van de Keere, F. and S. Tonegawa. 1998. CD4+ T cells prevent spontaneous experimental autoimmune encephalomyelitis in anti-myelin basic protein T cell receptor transgenic mice. J. Exp. Med. 188:1875-1882.

Wang, Y., I. Goldschneider, J. O'Rourke, and RE. Cone. 2001. Blood mononuclear cells induce regulatory NK T thymocytes in anterior chamber-associated immune deviation. J. Leukoc. Biol. 69:741-6.

Wilbanks, G. A. and J. W. Streilein. 1990. Characterization of suppressor cells in anterior chamber-associated immune deviation (ACAID) induced by soluble antigen. Evidence of two functionally and phenotypically distinct T-suppressor cell populations. Immunology 71:383-389.

Wilbanks, G. A. and J. W. Streilein. 1992. Fluids from immune privileged sites endow macrophages with the capacity to induce antigen-specific immune deviation via a mechanism involving transforming growth factor-beta. Eur. J. Immunol. 22:1031-1036.

Wilson, S. B., S. C. Kent, K. T. Patton, T. Orban, R. A. Jackson, M. Exley, S. Porcelli, D. A. Schatz, M. A. Atkinson, S. P. Balk, J. L. Strominger, and D. A. Hafler. 1998. Extreme Th1 bias of invariant Vα24JαQ T cells in type 1 diabetes [published erratum appears in Nature May 6, 1999 399(6731):84]. Nature 391:177-181.

Yoshimoto, T. and W. E. Paul. 1994. CD4⁺, NK1.1⁺ T cells promptly produce interleukin 4 in response to in vivo challenge with anti-CD3. J. Exp. Med. 179:1285-1295.

Yoshimoto, T., A. Bendelac, J. Hu-Li, and W. E. Paul. 1995. Defective IgE production by SJL mice is linked to the absence of CD4⁺, NK1.1⁺ T cells that promptly produce interleukin 4. Proc. Natl. Acad. Sci. U. S. A 92:11931-11934.

Zhang, B., T. Yamamura, T. Kondo, M. Fujiwara, and T. Tabira. 1997. Regulation of experimental autoimmune encephalomyelitis by natural killer (NK) cells. J. Exp. Med. 186:1677-1687.

While the present invention has been described in conjunction with a preferred embodiment, one of ordinary skill, after reading the foregoing specification, will be able to effect various changes, substitutions of equivalents, and other alterations to the compositions and methods set forth herein. It is therefore intended that the protection granted by Letters Patent hereon be limited only by the definitions contained in the appended claims and equivalents thereof.

What is claimed is:

1. A method of down-regulating an immune response in a patient, wherein said patient has an established immune-inflammatory condition, said method comprising the steps of:
(a) providing said patient;
(b) testing said patient for a response to an antigen;
(c) upon a positive response to said antigen, collecting antigen presenting cells from said patient;
(d) treating said antigen presenting cells with an inhibitory cytokine by incubation;
(e) incubating said treated antigen presenting cells with said antigen, thereby generating tolerogenic antigen presenting cells; and
(f) treating said patient with said tolerogenic antigen presenting cells, whereby said immune response is down-regulated in said patient.

2. The method of claim 1, wherein said immune-inflammatory condition is an autoimmune condition.

3. The method of claim 2, wherein said autoimmune condition is multiple sclerosis, pulmonary fibrosis, diabetes, or lupus.

4. The method of claim 1, wherein said immune-inflammatory condition is asthma or possible rejection following grafting, transplantation, or xenotransplantation.

5. The method of claim 1, wherein said immune-inflammatory condition is an allergic condition.

6. The method of claim 5, wherein said allergic condition is an allergy against Timothy grass pollen, dust, or dander.

7. The method of claim 1, wherein said inhibitory cytokine is transforming growth factor-β.

8. The method of claim 1, wherein said said inhibitory cytokine is interleukin-10.

9. The method of claim 1, wherein said said inhibitory cytokine is vasoactive intestinal peptide.

10. The method of claim 1, wherein said inhibitory cytokine is α-melanocyte stimulating hormone.

11. The method of claim 1, wherein said antigen presenting cells are dendritic cells.

12. The method of claim 1, wherein said antigen presenting cells are macrophages.

13. The method of claim 1, wherein said antigen presenting cells are B-cells.

14. The method of claim 1, wherein in the step of collecting said antigen presenting cells is from a population of cells from peripheral blood of said patient.

15. The method of claim 1, wherein in the step of collecting said antigen presenting cells is from a population of cells from bone marrow of said patient.

16. The method of claim 1, wherein said down-regulation of the immune response is for at least one year.

17. The method of claim 1, wherein said down-regulation of the immune response is for at least two years.

18. A method of down-regulating an immune response in a patient, wherein said patient has an established immune-inflammatory condition or is believed to be at risk of suffering from an immune-inflammatory condition, said method comprising the steps of:
(a) providing said patient;
(b) isolating a population of antigen presenting cells from said patient;
(c) treating said antigen presenting cells with an inhibitory cytokine by incubation;
(d) incubating said treated antigen presenting cells with an antigen relevant to said condition, thereby generating tolerogenic antigen presenting cells; and
(e) treating said patient with said tolerogenic antigen presenting cells.

19. The method of claim 18, wherein said inhibitory cytokine is transforming growth factor-β.

20. The method of claim 18, wherein said said inhibitory cytokine is interleukin-10.

21. The method of claim 18, wherein said said inhibitory cytokine is vasoactive intestinal peptide.

22. The method of claim 18, wherein said inhibitory cytokine is α-melanocyte stimulating hormone.

23. The method of claim 18, wherein said antigen presenting cells are dendritic cells.

24. The method of claim 18, wherein said antigen presenting cells are macrophages.

25. The method of claim 18, wherein said antigen presenting cells are B-cells.

26. The method of claim 18, wherein in said isolating step, the population of antigen presenting cells is from the peripheral blood of said patient.

27. The method of claim 18, wherein in said isolating step, the population of antigen presenting cells is from the bone marrow said patient.

28. The method of claim 18, wherein said immune-inflammatory condition is an autoimmune condition.

29. The method of claim 28, wherein said autoimmune condition is multiple sclerosis, pulmonary fibrosis, diabetes, or lupus.

30. The method of claim 18, wherein said immune-inflammatory condition is asthma or possible rejection following grafting, transplantation, or xenotransplantation.

31. The method of claim 18, wherein said immune-inflammatory condition is an allergic condition.

32. The method of claim 31, wherein said allergic condition is an allergy against Timothy grass pollen, dust, or dander.

33. The method of claim 18, wherein the immune response is down-regulated for at least one year.

34. The method of claim 18, wherein the immune response is down-regulated for at least two years.

35. A kit for making tolerogenic antigen presenting cells for treating an immune-inflammatory condition, said kit comprising:
(a) an inhibitory cytokine;
(b) an antigen relevant to said immune-inflammatory condition; and
(c) instructions for use in making tolergenic antigen presenting cells for the treatment of said immune-inflammatory condition.

36. The kit of claim 35, wherein said inhibitory cytokine is transforming growth factor-β.

37. The kit of claim 35, wherein said said inhibitory cytokine is interleukin-10.

38. The kit of claim 35, wherein said said inhibitory cytokine is vasoactive intestinal peptide.

39. The kit of claim 35, wherein said inhibitory cytokine is α-melanocyte stimulating hormone.

40. The kit of claim 35, wherein said immune-inflammatory condition is an autoimmune condition.

41. The kit of claim 40, wherein said autoimmune condition is multiple sclerosis, pulmonary fibrosis, diabetes, or lupus.

42. The kit of claim 35, wherein said immune-inflammatory condition is asthma or possible rejection following grafting, transplantation, or xenotransplantation.

43. The kit of claim 35, wherein said immune-inflammatory condition is an allergic condition.

44. The kit of claim 43, wherein said allergic condition is an allergy against Timothy grass pollen, dust, or dander.

45. A kit for making tolerogenic antigen presenting cells for treating multiple sclerosis, said kit comprising:
(a) transforming growth factor-β in a tolerogenic buffer;
(b) myelin basic protein; and
(c) instructions for use in making tolergenic antigen presenting cells for the treatment of multiple sclerosis.

46. An immunosuppressive agent for use during an immune-inflammatory condition, wherein said agent comprises a population of antigen presenting cells treated with an inhibitory cytokine and a relevant antigen, whereby said agent alters the reactivity of antigen presenting cells to become re-educated, thereby down-regulating an immune response in a patient with said immune-inflammatory condition.

47. The immunosuppressive agent of claim 46, wherein said inhibitory cytokine is transforming growth factor-β.

48. The immunosuppressive agent of claim 46, wherein said said inhibitory cytokine is interleukin-10.

49. The immunosuppressive agent of claim 46, wherein said said inhibitory cytokine is vasoactive intestinal peptide.

50. The immunosuppressive agent of claim 46, wherein said inhibitory cytokine is α-melanocyte stimulating hormone.

51. The immunosuppressive agent of claim 46, wherein said antigen presenting cells are mature dendritic cells.

52. The immunosuppressive agent of claim 46, wherein said antigen presenting cells are immature dendritic cells.

53. The immunosuppressive agent of claim 46, wherein said antigen presenting cells are macrophages.

54. The immunosuppressive agent of claim 46, wherein said antigen presenting cells are B-cells.

55. The immunosuppressive agent of claim 46, wherein said immune-inflammatory condition is an autoimmune condition.

56. The immunosuppressive agent of claim 55, wherein said autoimmune condition is multiple sclerosis, pulmonary fibrosis, diabetes, or lupus.

57. The immunosuppressive agent of claim 46, wherein said immune-inflammatory condition is asthma or possible rejection following grafting, transplantation, or xenotransplantation.

58. The immunosuppressive agent of claim 46, wherein said immune-inflammatory condition is an allergic condition.

59. The immunosuppressive agent of claim 58, wherein said allergic condition is an allergy against Timothy grass pollen, dust, or dander.

60. A method of treating a patient with an immune-inflammatory condition, wherein said immune-inflammatory condition is down-regulated, wherein said method comprises the steps of:
(a) providing said patient with said immune-inflammatory condition;
(b) administering to said patient an effective amount of the immunosuppressive agent of claim 46 in a pharmaceutically acceptable inert carrier substance.
